(19) United States
(12) Patent Application Publication
(10) Pub. No.: US 2008/0132565 A1
(43) Pub. Date: Jun. 5, 2008

(54) Polycyclic Macrolactones

(76) Inventors: Hans-Peter Fiedler, Tuebingen (DE); Roderich Suebmuth, Tuebingen (DE); Hana Zaehner, Bern (CH); Alan Bull, Canterbury Kent (GB)

Correspondence Address: FLYNN THIEL BOUTELL & TANIS, P.C., 2026 RAMBLING ROAD, KALAMAZOO, MI, 49008-1631, US

(21) Appl. No.: 10/574,237

(22) PCT Filed: Sep. 23, 2004
(86) PCT No.: PCT/EP04/10661

§ 371 Date: Aug. 23, 2007

(30) Foreign Application Priority Data
Oct. 1, 2003 (DE) 103 47 472.2
Nov. 11, 2003 (DE) 103 53 300.1

(51) Int. Cl. A61K 31/35; C07D 307/00; C12N 1/00; C12P 1/04; C12P 17/18; C12N 1/20; C07D 498/22

(52) U.S. Cl. 514/455; 549/265; 548/242; 435/243; 435/252.1; 435/41; 435/119

(57) ABSTRACT

The invention provides novel polycyclic macrolactones which can be produced, in particular, by a representative of the bacterial genus Verrucosispora. These substances are preferentially distinguished by their pharmacological effect. In particular, they exhibit an antibiotic effect. This antibiotic effect is preferably directed against Gram-positive bacteria.

### FIELD OF THE INVENTION

The invention relates to novel substances, to processes for preparing them, to the use of these substances and to pharmaceutical compositions.

### BACKGROUND OF THE INVENTION

Infectious diseases still constitute a very major medical problem world-wide. Of particular importance in this connection are the pathogen resistances which are increasingly occurring, particularly in the case of bacterial pathogens, with the resistances resulting in these pathogens no longer reacting to the drugs which are presently available. Bacteria which are resistant to a whole spectrum of active compounds are also appearing to an increasing extent, with these bacteria being referred to as being multiresistant pathogens. Many of the pathogenic multiresistant Gram-positive bacteria, such as the multiresistant and methicillin-resistant Staphylococcus aureus strains (MRSA), can currently only still be treated with glycopeptide antibiotics of the vancomycin/teicoplanin type. It is only a matter of time before Staphylococcus aureus strains whose multiresistance includes resistance to vancomycin appear in increasing numbers in the hospital environment. Such super-multiresistant strains have already been diagnosed in isolated instances and signify death for the infected patients since they cannot be treated.

### SUMMARY OF THE INVENTION

The invention is therefore based on the object of providing novel substances which are suitable for being used as active compounds for controlling pathogens, in particular bacterial pathogens, and can in this way be used as novel antibiotics. These novel substances are intended to be able to serve as model structures from which further active substances can subsequently be developed.

The substance according to the invention is characterized by the fact that it is a polycyclic macrolactone which can be produced by a representative of the bacterial genus Verrucosispora. This substance is advantageously secreted by the bacterium, i.e. meaning that it is released into the culture supernatant when the bacterium is cultured. Particular preference is given to this substance displaying a pharmacological effect and, in particular, an antibiotic effect. In a preferred embodiment, the substance according to the invention exhibits this antibiotic effect towards Gram-positive bacteria, in particular. In another preferred embodiment, the substance according to the invention exhibits a cytotoxic effect.

### BRIEF DESCRIPTION OF THE DRAWINGS

FIG. 1 shows a scanning electron microscopic photograph of the strain AB 18-032;

FIG. 2 shows the sequence of the 16S rRNA gene from the strain AB 18-032 (SEQ ID No. 1);

FIG. 3 shows the position of the strain AB 18-032 in the phylogenetic genealogical tree of the suborder Micromonosporineae (genealogical tree in accordance with Saitou, N. & M. Nei (1987), Mol. Biol. Evol. 4: 406-425);

FIG. 4 shows the pathways for the biosynthesis of para-aminobenzoic acid (left) and the biosynthesis of folic acid (right);

FIG. 5 shows UV spectra of the abyssomicins in accordance with the Formulae II, III and IV;

FIG. 6 shows the minimum inhibitory concentrations of the substance according to Formula III (abyssomicin C) when used against the multiresistant strains Staphylococcus aureus N135 and Staphylococcus aureus Mu50;

FIG. 7 shows (A) diaxial conformation of chorismate in aqueous solution; (B) configurational-structural formulae of the substances in accordance with the Formulae II, III and IV.

### DETAILED DESCRIPTION OF THE INVENTION

The inventors have been able to obtain preferred embodiments of the substance according to the invention by isolating and characterizing a novel bacterial strain belonging to the genus Verrucosispora. This strain, which is designated AB 18-032 in that which follows, was isolated from an ocean sediment which was collected from a depth of 1 000 m in Sagami-Bay in the Sea of Japan. The strain has been deposited in the Deutsche Sammlung von Mikroorganismen und Zellkulturen [German collection of microorganisms and cell cultures] GmbH (DSMZ) under the DSM No. 15899. Particular preference is therefore given to it being possible to prepare the substances according to the invention from the bacterial strain AB 18-032.

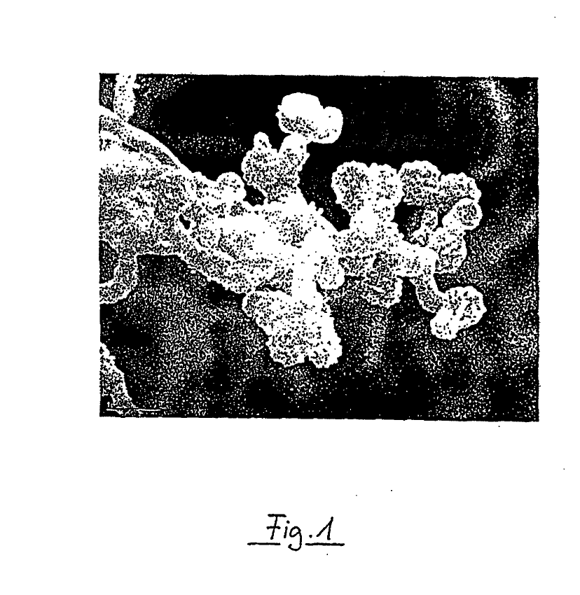

The morphological characteristics of the bacterial strain AB 18-032 can be described as follows. The strain grows as a surface culture on standard complex agar media, such as ISP-2 complex medium (0.4% yeast extract, 1% malt extract, 0.4% glucose, 1.5% agar), as orange-red colonies which turn black, due to sporulation, after an approx. two-week incubation at 27° C. FIG. 1 shows a scanning electron microscopic photograph of the sporulated substrate mycelium. The chemotaxonomic properties of strain AB 18-032 are summarized in Table 1.

TABLE 1
Chemotaxonomic characterization of strain AB 18-032

| | |
|---|---|
| Peptidoglucan | meso-diaminopimelic acid, the acyl |
| | type of the muramic acid is glycolyl- |
| | (Alγ′) |
| Total cell | Xylose |
| sugars | Mannose |
| Isoprenoid | MK9 (H₄) as main menaquinone |
| quinones | |
| Polar lipids | Type II (presence of diphosphatidyl- |
| | ethanolamine) |

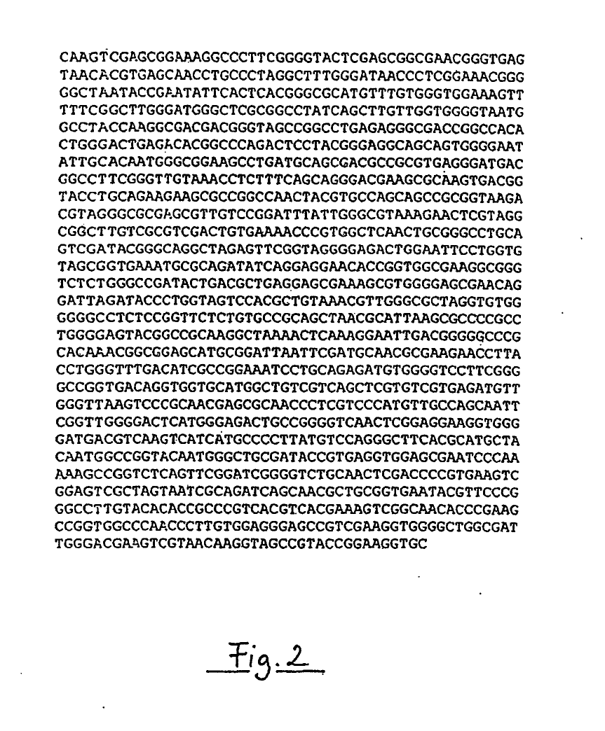

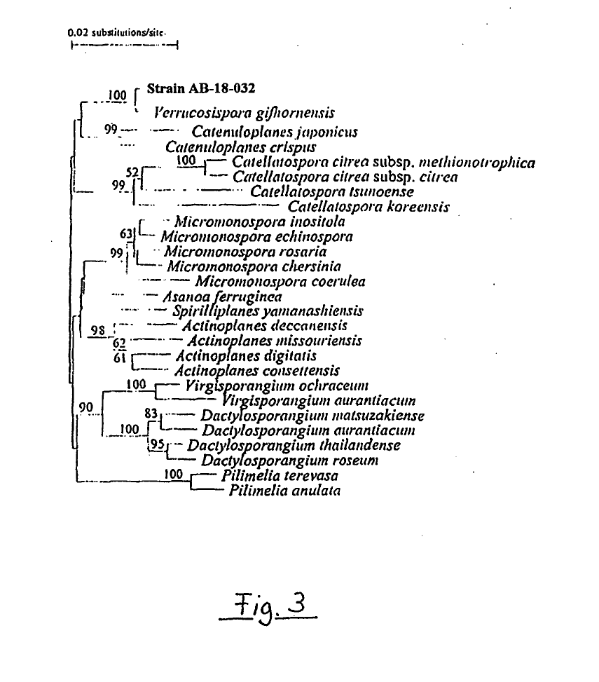

For a precise phylogenetic assignment, the complete nucleic acid sequence of the gene for the 16S ribosomal RNA was determined by direct sequencing of the PCR-amplified 16S rDNA [Chun, J. & M. Goodfellow (1995), Int. J. Syst. Bacteriol. 45: 240-245; Kim, S. B., C. Falkoner, S. T. Williams & M. Goodfellow (1998), J. Syst. Bacteriol. 48: 59-88]. After that, the sequence data were compared with the known sequences of representatives of the suborder Micromonosporineae. The highest degree of agreement of the AB 18-032 sequence, at 99.65%, was found to be with Verrucosispora gifhornensis. FIG. 2 depicts the sequence of the gene for the AB 18-032 16S rRNA (SEQ ID No. 1). The position of the strain of this invention on the Micromonosporineae phylogenetic genealogical tree, as shown in FIG. 3, was determined by comparing the sequence data for the AB 18-032 strain with the known 16S rRNA sequences of representatives of the suborder Micromonosporineae. On the basis of the phylogenetic analysis of the 16S rRNA and the above-described morphological and chemotaxonomic properties, it was possible to assign the strain AB 18-032 to the rare actinomycete genus Verrucosispora. This strain is the first marine representative of this genus and the second species of this genus which has thus far been described in the literature.

In order to characterize the novel strain AB 18-032 still further, its phenotypic properties were investigated in comparison with those of the known strain Verrucosispora gifhornensis [DSM 44337; Rheims, H., P. Schumann, M. Rohde & E. Stackebrandt (1998), Int. J. Syst. Bacteriol. 48: 1119-1127]. The results are summarized in Table 2.

TABLE 2
Phenotypic properties of the strain AB 18-032 and its closest
phylogenetic relative Verrucosispora gifhornensis DSM 44337

| | |
|---|---|
| Verrucosispora | |
| gifhornensis¹ | |
| DSM 44337 | Strain AB 18-032 |

Carbon utilization:

| | | |
|---|---|---|
| D(+)xylose | ++ | − |
| D(−)ribose | − | − |
| D-fructose | − | − |
| D(+)glucose | ++ | + |
| D(+)galactose | + | − |
| D-mannose | ± | ++ |
| maltose | + | ++ |
| saccharose | + | ++ |
| D(+)α-trehalose | + | ++ |
| L(+)arabinose | ++ | ++ |
| L(−)rhamnose | − | − |
| L(−)sorbose | − | − |
| α-lactose | − | + |
| α-melibiose | − | + |
| D(+)melezitose | − | + |
| D(+)raffinose | − | + |
| glycerol | − | + |
| dulcitol | − | + |
| meso-inositol | − | + |
| D-sorbitol | − | + |
| D-mannitol | − | + |
| salicin | + | + |

Nitrogen utilization:

| | | |
|---|---|---|
| DL-serine | + | ++ |
| L-aspartic acid | + | + |
| L-glutamic acid | + | ++ |
| L-histidine | + | + |
| L-arginine | + | ++ |
| L-valine | ± | ++ |
| L-methionine | − | ++ |
| L-phenylalanine | + | ++ |
| L-tryptophan | − | ++ |
| casein | p | p |
| cellulose breakdown | n | n |
| gelatin liquefaction | p | nd |
| formation of nitrate | n | n |
| from nitrite | | |
| starch hydrolysis | p | p |

¹Data from Rheims et al. (1998)
++, good utilization; +, normal utilization; ±, poor utilization;
−, no utilization; p, positive; n, negative; nd, not determined

This representative of the genus Verrucosispora, which the inventors have isolated and characterized for the first time, produces a variety of substances which advantageously display a pharmacological effect. These substances are summarized below under the designation abyssomicins.

In a preferred embodiment of the invention, the substances according to the invention are characterized by the general Formula I

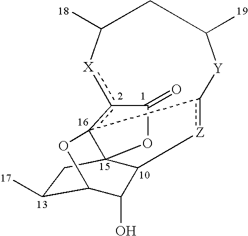

### In this formula,

### X is C═O or

C—OH

### Y is

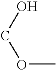

or

C═O

### Z is C═N— or

CH or

CH₂

The dotted lines indicate bonds which may be present. The numerals denote the numbering of the carbon atoms in the skeleton used for assigning the ¹H and ¹³C chemical shifts in the NMR analysis. The Formula I is representative of all the conceivable relative configurations and encompasses all the possible stereoisomers.

This general formula encompasses different substances, that is polycyclic macrolactones, which can particularly advantageously be employed as active compounds directed against microorganisms, in particular against bacteria and/or protozoa. The structure of these substances, that is of the abyssomicins, constitutes a novel model structure which can be used to develop novel antibiotically active substances.

A preferred embodiment of the substances according to the invention can be depicted by the following Formula II

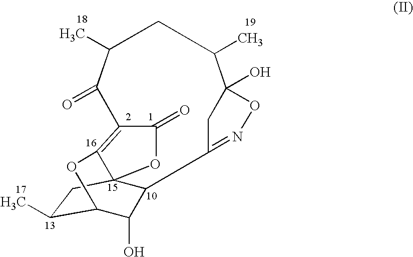

### This formula, and also all the other formulae which are listed here, are representative of all the possible relative configurations, for example of the mirror-image Formula IIa as well.

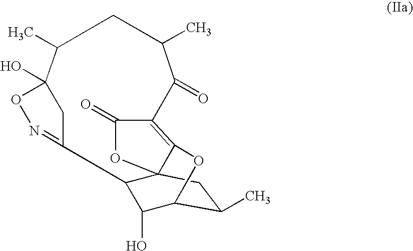

Another preferred embodiment can be described by the Formula IV

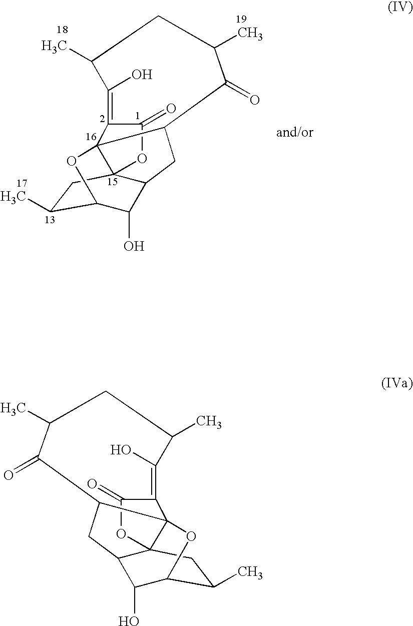

which likewise encompasses all the possible configurations.

A very particularly preferred embodiment of the substances according to the invention is characterized by the Formula III

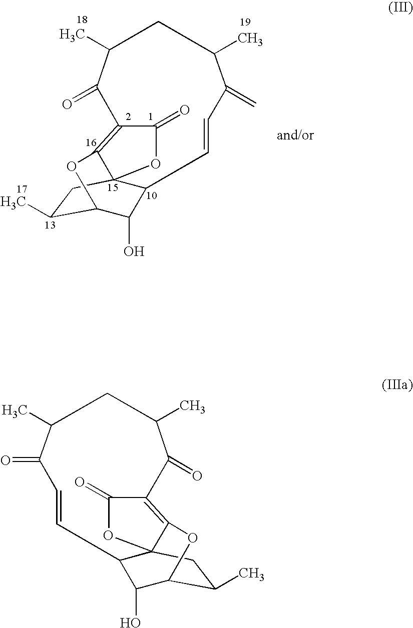

together with in each case all the possible relative configurations. In particular, this embodiment is characterized by particularly advantageous antibiotic properties which are displayed, in particular, towards Gram-positive bacteria. In that which follows, the substance in accordance with Formula II is designated abyssomicin B, while the substance in accordance with Formula III is designated abyssomicin C and the substance in accordance with Formula IV is designated abyssomicin D.

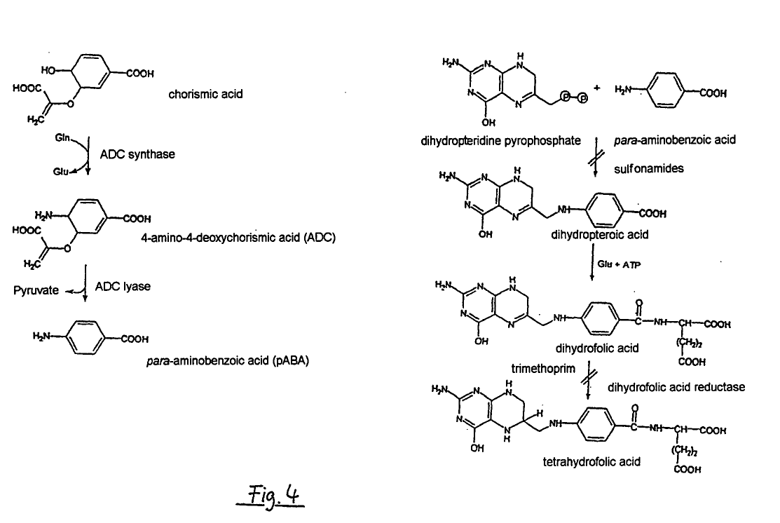

The invention also encompasses substances which are characterized by the fact that they inhibit the biosynthesis of para-aminobenzoic acid (pABA). In particular, these substances according to the invention inhibit the synthesis of para-aminobenzoic acid from chorismic acid. For illustrative purposes, the pathway of the biosynthesis of para-aminobenzoic acid from chorismic acid is shown on the left-hand side in FIG. 4. Para-aminobenzoic acid is an essential building block in folic acid biosynthesis, which is shown on the right-hand side in FIG. 4. The substances according to the invention therefore ultimately inhibit the synthesis of folic acid. The latter is a vitamin which is essential to the life of microorganisms, in particular prokaryotes and protozoa, which means that their metabolism is impaired by the substances according to the invention such that the latter can be used to control the corresponding microorganisms. The particular advantage of this point of attack by the substances according to the invention is that mammals, and in particular humans, do not possess this pathway for biosynthesizing folic acid which means that mammalian cells, in particular, are not negatively affected by the substances according to the invention. As a consequence, the substances according to the invention can, for example, be used for treating diseases, in particular infectious diseases, in humans or animals without displaying any extensive side effects. In a particularly preferred embodiment of these substances, the latter exhibit features in accordance with the above description.

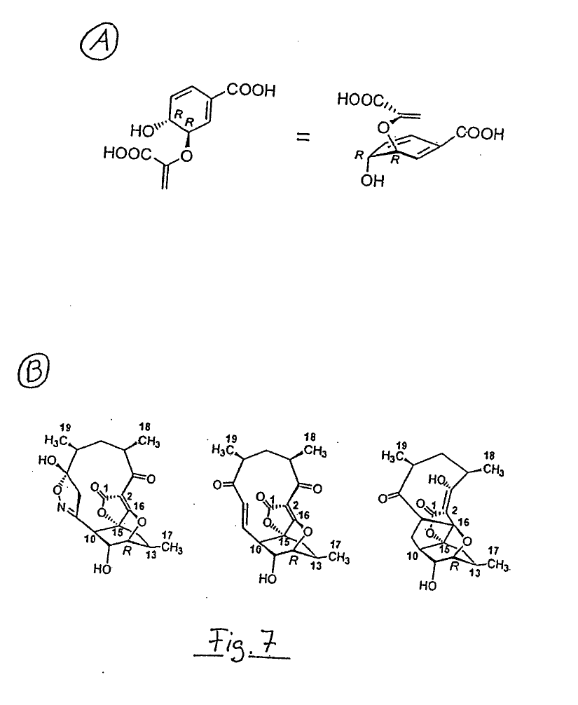

The invention furthermore encompasses polycyclic macrolactones as substances which, as constituent structures, exhibit at least one oxabicyclo system and at least one Michael system as double bond system. The Michael system is preferably a trans double bond which is in conjugation with a ketone. Particular preference is given to this Michael system being, for example, located at positions C7 to C9 in a ring system in accordance with the general Formula I. Experiments carried out by the inventors have shown that such a Michael system can advantageously be directly involved in the mechanism of action of the substances according to the invention by, for example, advantageously interacting irreversibly with nucleophilic amino acid side chains. The oxabicyclo system which, according to the invention, is present in the polycyclic macrolactones exhibits similarities with the solution conformation of chorismate. The corresponding conformations of chorismate are shown for illustration in FIG. 7A. The substances according to the invention can therefore in a certain manner imitate the substrate chorismate such that this thereby makes it possible to explain the particular effect of the substances according to the invention. This oxabicyclo system can, for example, be configured as can be seen from the Formulae I to IV. Particular preference is given to such a bicyclo system being located in the vicinity of the described Michael system. A preferred embodiment of such a substance, which exhibits a Michael system and an oxabicyclo system, can be described, for example, by the Formula III.

Preference is furthermore given to position C12 in the exemplary substances in accordance with Formulae I to IV exhibiting an (R) configuration. For illustration, reference is made to FIG. 7B, which shows examples of the substances according to the invention in corresponding configuration, with the formulae which are shown here corresponding, from left to right, to the Formulae II, III and IV.

The invention furthermore encompasses substances which are characterized by the fact that they are derivatives of the above-described polycyclic macrolactones. These substances can be naturally occurring substances. On the other hand, the derivatives also include substances which are at least in part synthetic or else have been prepared using other means and can, for example, be derived from naturally occurring substances. Thus, the above-described substances can be used as model structures for the purpose of designing and preparing correspondingly suitable substances which may possibly exhibit additional advantages as compared with the starting substances. These suitable substances can advantageously be antibiotically active substances which have an antibiotic activity which is similar or superior to that of the starting substance but which exhibit better properties than the starting substances, for example with regard to side effects in an organism or bioavailability in an organism. The reader is referred to the above description with regard to other features of these substances according to the invention.

In another aspect, the invention encompasses pharmaceutical compositions which comprise at least one substance in accordance with the above description and at least one pharmaceutically acceptable excipient. In particular, the invention encompasses pharmaceutical compositions which, in addition to at least one pharmaceutically acceptable excipient, comprise at least one substance which inhibits the biosynthesis of para-aminobenzoic acid and, in particular, the synthesis of para-aminobenzoic acid from chorismic acid. These pharmaceutical compositions can advantageously be used to control microorganisms and, in particular, bacteria and/or protozoa.

Particularly advantageously, these pharmaceutical compositions can be used for treating infectious diseases which are caused by bacteria or are at least influenced by bacteria. Very particular preference is given to these pharmaceutical compositions being used for controlling Gram-positive bacteria. The pharmaceutical compositions are furthermore also suitable for treating infectious diseases which are caused, or at least influenced, by other microorganisms, for example protozoa. Examples of infectious protozoa which can be controlled using the substances according to the invention are plasmodias, leishmanias and trypanosomes, which are responsible for tropical infectious diseases (malaria, leishmaniasis, African sleeping sickness and Chagas disease). The particularly advantageous effect of these pharmaceutical compositions or of the corresponding substances is based, especially, on the fact that these substances ultimately inhibit the biosynthesis of folic acid. This metabolic pathway is only present in the microorganisms, in particular bacteria and/or protozoa, to be controlled and not in animals or humans, which can be treated with these compositions. It is particularly advantageous to be able to use these compositions to control microorganisms which are hospital pathogens, in particular pathogenic multiresistant bacteria which no longer respond to conventional antibiotics. With very great advantage, the pharmaceutical compositions are suitable for treating infectious diseases which are at least concomitantly influenced by Gram-positive bacteria. For example, it is possible to use the pharmaceutical compositions according to the invention to control multiresistant and, in particular, methicillin resistant, Staphylococcus aureus strains (MRSA). It is also possible, for example, to treat infectious diseases which involve Staphylococcus aureus strains which, in addition to various other resistances, are also resistant to vancomycin. Resistances to vancomycin have already been diagnosed on various occasions. Particularly in such a case, treatment with the pharmaceutical compositions according to the invention may protect a patient from death since there is otherwise no possibility of treating such super-multiresistant strains. It is naturally also possible to use the pharmaceutical compositions for controlling pathogenic microorganisms which have not developed any resistances, or only a few resistances, to conventional antibiotics.

The invention also encompasses the use of the above-described substances for treating infectious diseases which are at least concomitantly influenced by bacteria and/or protozoa. The invention furthermore encompasses a use of the substances according to the invention for producing a drug for treating infectious diseases which are at least concomitantly influenced by bacteria and/or protozoa. The invention also encompasses the use of substances for treating said infectious diseases, with the substances inhibiting the synthesis of para-aminobenzoic acid and, in particular, the synthesis of para-aminobenzoic acid from chorismic acid. The use of corresponding substances for producing a drug for treating infectious diseases which are at least concomitantly influenced by bacteria and/or protozoa is likewise encompassed. The invention furthermore encompasses a method for treating infectious diseases which are at least concomitantly influenced by bacteria and/or protozoa, with at least one substance being administered in the form of a pharmaceutical composition in accordance with the above description. The reader is referred to the above description with regard to other features of these different uses and methods.

The invention furthermore encompasses a method for controlling microorganisms, in particular bacteria and/or protozoa, with use being made of at least one of the substances according to the invention which are described above. Such control of microorganisms can, for example, be a disinfection method. Particularly in hospitals and other medical institutions, it is absolutely necessary for surfaces of the widest possible variety, such as surfaces of surgical instruments or of equipment articles, to be disinfected in order to prevent any infection with disease-eliciting microorganisms. The substances according to the invention can very advantageously be employed in this connection, with this particularly preferably taking place in combination with other disinfectants.

The invention also encompasses a microorganism which is characterized by the fact that it is able to produce at least one substance as described above. In a preferred embodiment, the microorganism is a bacterium, with this bacterium preferably being a representative of the genus Verrucosispora. Particular preference is given to the bacterium in this connection being the bacterial strain AB 18-032 (DSM 15899). The bacterial strain AB 18-032 is the strain from which the inventors were able to isolate the substances which have been cited by way of example. Mutants of these microorganisms and, in particular, of the strain AB 18-032, are likewise encompassed by the invention. The invention also encompasses other microorganisms which produce corresponding substances. In this connection, particular preference is given to microorganisms which are able, for example, to produce relatively large quantities of the substances according to the invention. These microorganisms can particularly advantageously be employed for preparing the quantities of the substances according to the invention which are required for therapeutic uses.

Finally, the invention encompasses a process for preparing at least one substance according to the invention, in which process a microorganism which is able to produce at least one of the described substances is first of all cultured. The substance is preferably secreted by the microorganism such that a filtrate of the culture supernatant, in which the desired substance is present, is prepared in a following procedural step. This culture filtrate, or else the culture supernatant, can be used directly for the purpose of appropriately employing the substances according to the invention. On the other hand, the substances can also be isolated from the culture filtrate or the culture supernatant and preferably purified to a greater or lesser extent in order, in this way, to have the substance available in purified form. This is especially advantageous for medical applications since, if at all possible, only purified substances should be used for pharmaceutical purposes in order to avoid any undesirable effects due to the presence of other compounds. It may furthermore be preferable for the substance not to be secreted but, instead, to remain within the microorganism. In this case, the substance is isolated from the cultured microorganisms using suitable methods which are known to the skilled person. The microorganism which is used is advantageously the strain AB 18-032. It may, however, also be very advantageous to use a microorganism which is, for example, optimized with regard to the quantity of the substance to be produced. A corresponding optimization can be effected, for example, by means of appropriate selection. The microorganism is preferably cultured in the presence of a medium which at least contains a carbon source, a nitrogen source and mineral salts. While the substances are subsequently isolated with preference from the culture filtrate, they can also be directly isolated from the culture supernatant. The substances can, for example, be isolated from the culture filtrate or the supernatant by means of solvent (e.g. ethyl acetate) extraction. Another possibility is, for example, a column chromatography using a polystyrene resin (e.g. Amberlite XAD-16). A further isolation or purification can be effected by fractionating the different substances, for example by means of adsorption and/or exclusion chromatography. The substances can be obtained in pure form by crystallization. Where appropriate, the purified substances can be subjected to further reaction using conventional chemical methods. Details with regard to this preparation process will be immediately apparent to the skilled person.

Details with regard to the described features, and also other features of the invention, are given in the following description of examples taken in combination with the figures and the subclaims. In this connection, the individual features can in each case be realized individually or in combination with each other.

### EXAMPLES

### 1. Screening for Inhibitors of the Biosynthesis of Chorismic Acid, of Para-Aminobenzoic Acid (pABA) and of the Aromatic Amino Acids

Inhibitors of chorismic acid biosynthesis and of the biosynthesis pathways which derive from chorismic acid are determined using what is termed a cross test which is based on a modified agar diffusion test. The test organism which is used is Bacillus subtilis, which is embedded in a chemically defined agar medium. One of the filter paper strips of the cross test assay is saturated with a cell extract while the second filter paper strip contains one of the following variations: (a) Tyr+Phe+Trp+pABA, (b) Tyr+Phe, (c) Trp and (d) pABA. The missing pattern of the individual variants makes it possible to decide whether the inhibitor is an inhibitor of early aromatic compound biosynthesis (prior to chorismic acid) or an inhibitor which intervenes after chorismic acid and, in this case, whether the inhibitor is an inhibitor of tyrosine (Tyr)/phenylalanine (Phe) biosynthesis, of tryptophan (Trp) biosynthesis or of para-aminobenzoic acid (pABA) biosynthesis. Only the extract whose inhibitory effect is antagonized by (a) and (d) contains an antagonist of pABA which inhibits pABA biosynthesis after chorismic acid. This test was used to find strain AB 18-032, which produces the active substances.

### 2. Production of the Polycyclic Macrolactones by the Bacterial Strain AB 18-032

The strain AB 18-032 produces the polycyclic macrolactones during its logarithmic and through into its stationary growth phases. A typical fermentation is carried out in the following manner: a 10 liter blade mixer fermenter is filled with 9.5 liters of complex medium (1% glucose, 1% starch, 1% glycerol, 0.25% corn steep powder, 0.5% peptone, 0.2% yeast extract, 0.1% NaCl, 0.3% CaCO₃; pH 7.3). The fermenter is inoculated with 5% by volume of a 48-hour shaken culture (500 ml Erlenmeyer flask having a lateral stab hole, 100 ml of complex medium, 120 rpm, 27° C.). The fermenter is incubated for 4-5 days at 27° C., a rotational speed of 200 rpm and an aeration rate of 0.5 vvm. The polycyclic macrolactones can be detected in the culture supernatant by means of HPLC diode array detection (HPLC-DAD) and biological testing.

### 3. Isolation of the Polycyclic Macrolactones

The fermentation broth is separated into biomass and culture filtrate in the added presence of 2% filtration aid (Highphlo Supercel). The biomass is discarded. The culture filtrate is adjusted to pH 4 (HCl) and extracted 2× with in each case ¼ of the volume of ethyl acetate. The organic phases are combined and concentrated down to an oily residue on a vacuum rotary evaporator. The oily residue is extracted 2× with a small volume of petroleum benzine in order to remove fats. The petroleum benzine extract is discarded.

The oily residue is dissolved in a little methanol and fractionated into the individual crude substance fractions on a Sephadex LH-20 column (100×5 cm) in methanol. Pure polycyclic macrolactones are isolated by means of low-pressure chromatography on a LiChroprep diol column (40×2.6 cm), using a linear gradient of dichloromethane to dichloromethane/methanol (90+10) in 3 hours and at a flow rate of 2 ml/min, and subsequent exclusion chromatography on a Fractogel TSK HW 40 column (90×2.5 cm) in methanol at a flow rate of 0.5 ml/min.

### 4. HPLC-DAD Analysis of the Polycyclic Macrolactones

Chromatographic equipment: HP 1090 liquid chromatograph with integrated diode array detection system and HP Kayak XM 600 ChemStation with HPLC software A.08.03 (Agilent Technologies). The multichannel detection took place at 210, 230, 260, 280, 310, 360, 435 and 500 nm, while the UV-vis spectra were recorded at 200-600 nm.

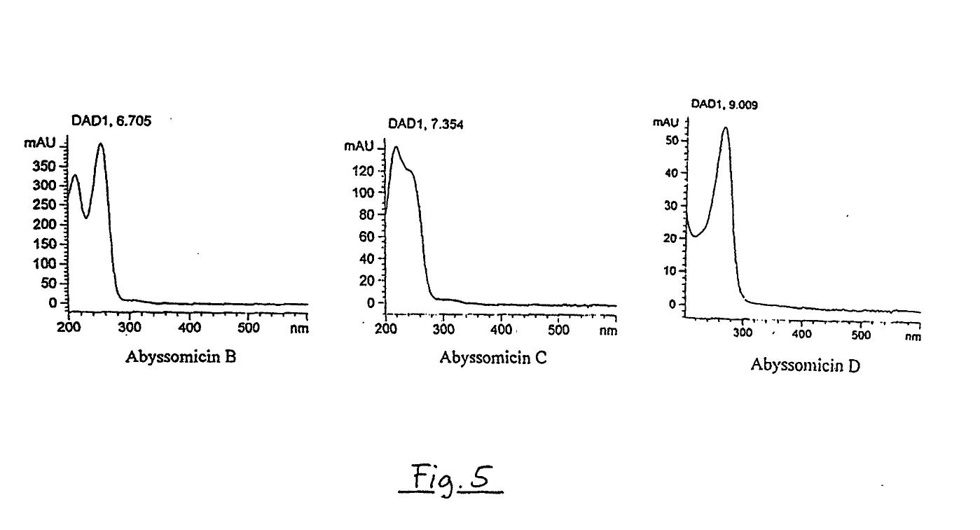

Separation parameters: HPLC column filled with Nucleosil-100-18 (125×4.6 mm, precolumn 20×4.6 mm, particle size 5 μm; Macherey & Nagel). Linear gradient elution from 100% aqueous phosphoric acid (0.1% v/v) to 100% acetonitrile in 15 min at a flow rate of 2 ml/min. The injection volume is 10 μl. The retention times are 6.7 min for abyssomicin B, 7.35 min for abyssomicin C and 9.0 min for abyssomicin D. Aside from their retention times, the abyssomicins are identified on the basis of their characteristic UV spectra (FIG. 5).

### 5. Structure Elucidation

LC-MS experiments: Agilent 1100 HPLC system (Agilent Technologies) coupled to a Bruker Esquire 3000+ mass spectrometer (Bruker-Daltonics).

ESI-FTICR-MS: the mass spectra were recorded on an APEX II FTICR mass spectrometer (4.7 T; Bruker-Daltonics). PEG 400 was used for the internal calibration.

¹H/¹³C NMR experiments (1D: 1H, 2D: COSY, TOCSY, HSQC, HMBC) were carried out on an AMX 600 NMR spectrometer (Bruker) with 5 mm triple resonance sample head using Z gradients.

### 6. Physicochemical Properties

The isolated substances exhibited the following physicochemical properties:

### Abyssomicin B:

### Colorless substance, soluble in methanol and DMSO

Empirical formula: C₁₉H₂₃NO₇[377]

### ESI-FTICR-MS: [M+Na]⁺=400.13654 Da

### ([M+Na]⁺_{theor.}=400.13667 Δm=0.34 ppm; C₁₉H₂₃NO₇Na)

¹H NMR/¹³C NMR data: see Table 3

TABLE 3
¹H NMR and ¹³C NMR chemical shifts of abyssomicin B
([D6] DMSO, 305K); coupling constants not determined

| | | |
|---|---|---|
| | ¹H δ | ¹³C δ |
| No. | [ppm] | [ppm] |

| 1 | — | | 169.4 |
|---|---|---|---|
| 2 | — | | 99.7 |
| 3 | — | | 212.6 |
| 4 | 3.18 | | 41.9 |
| 5 | 2.59 | (a) | 34.8 |
| | 2.59 | (b) | |
| 6 | 2.14 | | 43.7 |
| 7 | — | | 197.1 |
| 8 | 1.33 | (a) | 38.5 |
| | 1.77 | (b) | |
| 9 | — | | n.d. |
| 10 | 2.59 | | 45.8 |
| 11 | 4.24 | | 68.9 |
| | 5.82 | (OH) | — |
| 12 | 4.55 | | 84.5 |
| 13 | 2.55 | | 24.2 |
| 14 | 2.54 | (a) | 36.9 |
| | 1.04 | (b) | |
| 15 | — | | 78.0 |
| 16 | — | | 184.5 |
| 17 | 0.99 | | 18.7 |
| 18 | 0.97 | | 16.6 |
| 19 | 0.96 | | 20.1 |

n.d. = not determined

### Abyssomicin C

### Colorless substance, soluble in methanol and DMSO

Emperical formula: C₁₉H₂₄O₆[346]

### ESI-FTICR-MS: [M+Na]⁺=369.13079 Da

### ([M+Na]⁺_{theor.}=369.13085 Δm=0.20 ppm; C₁₉H₂₂O₆Na)

¹H NMR/¹³C NMR data: see Table 4

TABLE 4
¹H NMR and ¹³C NMR chemical shifts of abyssomicin C
([D4]MeOD, 298K)

| | ¹H δ | | | ¹³C δ |
|---|---|---|---|---|
| No. | [ppm] | | Coupling constants [Hz] | [ppm] |
| 1 | — | | — | 173.8 |
| 2 | — | | — | 106.7 |
| 3 | — | | — | 202.8 |
| 4 | 3.51 | | m ³J_{4, 18} = 6.7; ³J_{4, 5a} = 11.2; ³J_{4, 5b} = 2.7 | 45.3 |
| 5 | 2.01 | (a) | m ²J_{5a, 5b} = 14.1; ³J_{5a, 4} = 11.2; | 43.2 |
| | | | ³J_{5a, 6} = 10.1 | |
| | 1.44 | (b) | m ²J_{5b, 5a} = 14.1; ³J_{5b, 4} = 2.7; | |
| | | | ³J_{5b, 6} = 1.6 | |
| 6 | 2.94 | | m ²J_{6, 5a} = 10.1; ³J_{4, 5b} = 1.6; ³J_{6, 19} = 7.2 | 50.3 |
| 7 | — | | — | 208.4 |
| 8 | 6.55 | | m ³J_{8, 9} = 13.5 | 137.1 |
| 9 | 5.98 | | dd ³J_{9, 8} = 13.5; ³J_{9, 10} = 9.5 | 137.3 |
| 10 | 2.99 | | dd ³J_{10, 9} = 9.5; ³J_{10, 11} = 6.1 | 51.5 |
| 11 | 5.06 | | dd ³J_{11, 10} = 6.1; ³J_{11, 12} = 3.3 | 76.0 |
| | 4.59 | (OH) | — | — |
| 12 | 4.57 | | d ³J_{12, 11} = 3.3; ³J_{12,13} = n.d. | 88.9 |
| 13 | 2.73 | | n.d. | 28.1 |
| 14 | 1.26 | (a) | dd ²J_{14a, 14b} = 12.4; ³J_{14a, 13} = 23.9 | 39.6 |
| | 2.69 | (b) | dd ²J_{14, 14a} = 12.4; ³J_{14b, 13} = 2.4 | |
| 15 | — | | — | 81.1 |
| 16 | — | | — | 189.8 |
| 17 | 1.17 | | d ³J_{17, 13} = 17.0 | 21.5 |
| 18 | 1.09 | | d ³J_{18, 4} = 6.7 | 19.3 |
| 19 | 1.11 | | d ³J_{19, 6} = 7.2 | 23.0 |

n.d. = not determined

### Abyssomicin D:

### Colorless substance, soluble in methanol and DMSO

### Emperical formula C₁₉H₂₄O₆[348]

### ESI-FTICR-MS: [M+Na]⁺=371.14663 Da

### ([M+Na]⁺_{theor.}=371.14650 Δm=0.32 ppm; C₁₉H₂₄O₆Na)

¹H NMR/¹³C NMR data: see Table 5

TABLE 5
¹H NMR and ¹³C NMR chemical shifts of abyssomicin D
([D6]DMSO, 305K)

| | ¹H δ | | | ¹³C δ |
|---|---|---|---|---|
| No. | [ppm] | | Coupling constants [Hz] | [ppm] |
| 1 | — | | — | 172.9 |
| 2 | — | | — | 98.0 |
| 3 | — | | — | 178.9 |
| | 11.04 | (OH) | — | — |
| 4 | 2.42 | | m n.d. | 39.5 |
| 5 | 1.59 | (a) | dd ²J_{5a, 5b} = 15.0; ³J_{5a, 4} = n.d.; | 32.5 |
| | | | ³J_{5a, 6} = n.d. | |
| | 2.70 | (b) | dd ²J_{5b, 5a} = 15.0; ³J_{5b, 4} = n.d.; | |
| | | | ³J_{5b,6} = n.d. | |
| 6 | 2.14 | | m n.d. | 47.3 |
| 7 | — | | — | 210.3 |
| 8 | 3.57 | | t ³J_{8, 9a} = 8.03; ³J_{8, 9b} = 9.8 | 57.8 |
| 9 | 1.54 | (a) | dd ³J_{9a, 9b} = 12.0; ³J_{9a, 8} = 8.03; | 26.1 |
| | | | ³J_{9b, 10} = n.d. | |
| | 2.00 | (b) | m ³J_{9b, 9a} = 12.0; ³J_{9b, 10} = 3.5; | |
| | | | ³J_{9b, 8} = 9.8 | |
| 10 | 2.26 | | d J_{10b, 9b} = 3.5; ³J_{10, 11} = n.d.; | 47.5 |
| | | | ³J_{10, 9a} = n.d. | |
| 11 | 4.09 | | d ³J_{11, 12} = 4.0; ³J_{11, 10} = n.d. | 72.1 |
| | 5.53 | (OH) | — | — |
| 12 | 3.54 | | d ³J_{12, 11} = 4.0; ³J_{12, 13} = n.d. | 76.0 |
| 13 | 2.46 | | dd n.d. | 23.7 |
| 14 | 2.29 | (a) | dd n.d. | 31.8 |
| | 0.91 | (b) | dd n.d. | |
| 15 | — | | — | 86.9 |
| 16 | — | | — | 84.5 |
| 17 | 0.93 | | d ³J_{17, 13} = 6.8 | 18.0 |
| 18 | 1.27 | | d ³J_{18, 4} = 7.4 | 18.7 |
| 19 | 1.01 | | d ³J_{19, 6} = 7.1 | 18.3 |

n.d. = not determined

### 7. Antibiotic Activity in the Agar Diffusion Test and Activity Spectrum

In the agar diffusion test, abyssomicin C exhibits an antibiotic effect which is directed, in particular, against the Gram-positive bacteria which are tested. The Gram-negative bacteria and fungi which were tested were insensitive to the abyssomicins. The antibiotic activity spectrum is shown in Table 6.

TABLE 6
Antibiotic activity of abyssomicin C in the agar
diffusion test (10 μl of antibiotic solution per
filter disk; inhibition zone diameters in mm)

| | | Abyssomicin | | |
|---|---|---|---|---|
| | | C (mg/ml) | | |
| Test organism | Medium | 1 | 0.3 | 0.1 |
| Arthrobacter aurescens DSM 20166 | CM | 14 | 10 | — |
| Brevibacillus brevis DSM 30 | CM | 17 | 12 | 9 |
| Brevibacillus brevis DSM 30 | MM | 30 | 24 | 19 |
| Bacillus subtilis DSM 10 | CM | 16 | 12 | 10 |
| Bacillus subtilis DSM 10 | MM | 26 | 19 | 14 |
| Micrococcus luteus ATCC 381 | CM | — | — | — |
| Mycobacterium phlei DSM 750 | CM | — | — | — |
| Staphylococcus aureus DSM 20231 | CM | 19 | 11 | 9 |
| Rhodococcus erythropolis DSM 1069 | CM | 27 | 18 | 15 |
| Rhodococcus erythropolis DSM 1069 | CM | 30 | 28 | 21 |
| Streptomyces viridochromogenes Tü 57 | CM | 11 | 9 | — |

CM, complex medium; MM, chemically defined medium

### 8. Minimum Inhibitory Concentration

The minimum inhibitory concentration (MIC) of abyssomicin C was determined in a dilution series test. The results are shown in Table 7. As expected, the test organisms are substantially more sensitive to abyssomicin C in chemically defined medium.

TABLE 7
Minimum inhibitory concentration (MIC; μg/ml) of abyssomicin C in
a dilution series test (2 ml test tube scale, shaker at 120 rpm)

| Test organism | Medium | MIC |
|---|---|---|
| Bacillus subtilis DSM 10 | CM | 10 |
| Bacillus subtilis DSM 10 | MM | 0.1 |
| Rhodococcus erythropolis DSM 1069 | CM | 10 |
| Staphylococcus aureus DSM 20231 | CM | 100 |

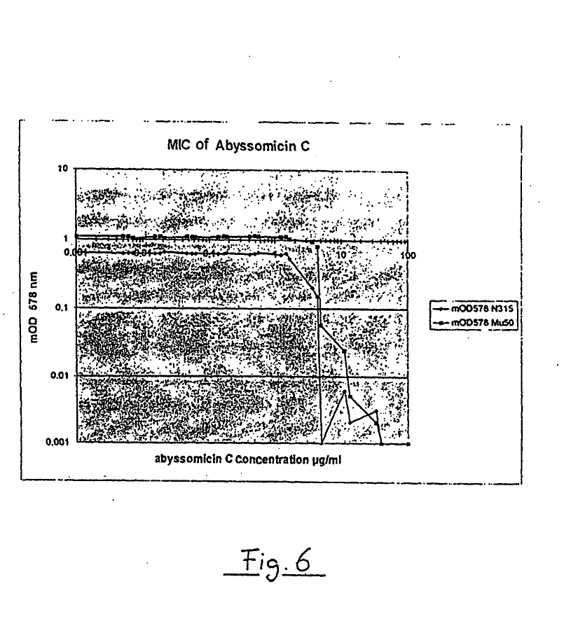

The MIC with regard to clinically pathogenic Staphylococcus aureus strains was determined in a microtiter plate assay. The activity of abyssomicin C toward the multiresistant strain S. aureus N315, whose multiresistance includes resistance to methicillin, was determined, as was the activity of abyssomicin C toward the multiresistant strain S. aureus Mu50, whose multiresistance includes resistance to vancomycin. The results are shown in FIG. 6.

1. A polycyclic macrolactone which is produced by a representative of the bacterial genus Verrucosispora.

2. A substance exhibiting a pharmacological effect, comprising the polycyclic macrolactone of claim 1.

3. A substance exhibiting an antibiotic effect towards Gram-positive bacteria, comprising the polycyclic macrolactone of claim 1.

4. The polycyclic macrolactone as claimed in claim 1, wherein the representative of the bacterial genus Verrucosispora is bacterial strain AB 18-032 (DSM 15899).

5. The polycyclic macrolactone of claim 1, characterized by having the structure of Formula (I) 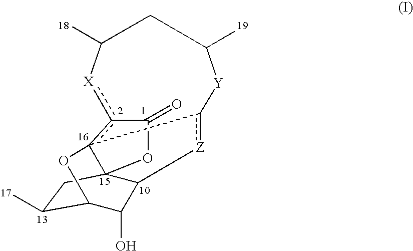
where X is C═O or C—OH,
Y is 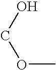
or C═O
Z is C═N—, CH or CH₂.

6. The polycyclic macrolactone of claim 5, characterized by having the structure of Formula II

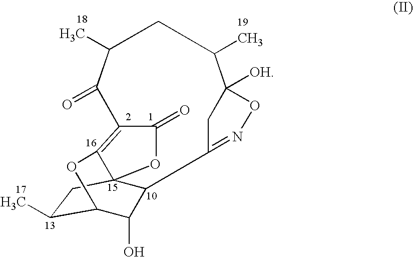

7. The polycyclic macrolactone of claim 5, characterized by having the structure of Formula III

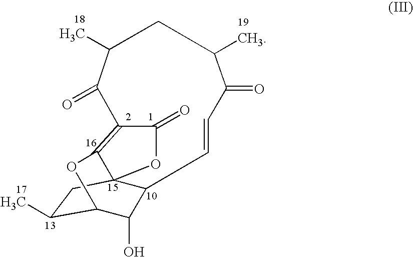

8. The polycyclic macrolactone of claim 5, characterized by having the structure of Formula IV

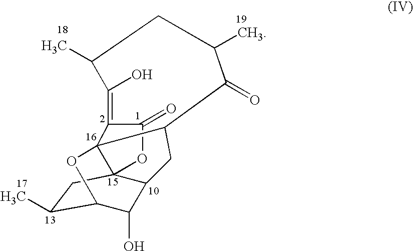

9. A substance which inhibits the synthesis of para-aminobenzoic acid from chorismic acid, comprising the polycyclic macrolactone of claim 1.

10. The polycyclic macrolactone of claim 1, containing at least one oxabicyclo system and at least one Michael system as a double bond system.

11. (canceled)

12. A pharmaceutical composition comprising at least one polycyclic macrolactone as claimed in claim 1 and at least one pharmaceutically acceptable excipient.

13. A pharmaceutical composition comprising at least one substance which inhibits the synthesis of para-aminobenzoic acid from chorismic acid and at least one pharmaceutically acceptable excipient.

14. A method of treating infectious diseases in a subject which are at least concomitantly influenced by bacteria and/or protozoa, comprising the step of administering to the subject the polycyclic macrolactone of claim 1.

15.-21. (canceled)

22. A microorganism, characterized in that it is able to produce at least one substance as claimed in claim 1.

23. A microorganism as claimed in claim 22, characterized in that it is a strain of the bacterial genus Verrucosispora, or a mutant thereof.

24. A microorganism, in particular as claimed in claim 22, characterized in that it is the strain AB 18-032 (DSM 15899) of the bacterial genus Verrucosispora, or a mutant thereof.

25. A process for preparing at least one substance comprising the procedural steps of:
a) culturing a microorganism as claimed in claim 22,
b) obtaining a culture supernatant from the culture,
c) where appropriate, preparing a culture filtrate, and
d) where appropriate, isolating one or more substances from the culture supernatant and/or the culture filtrate.

26. A process for preparing the at least one substance as claimed in claim 22, comprising the procedural steps of:
a) culturing the microorganism, and
b) isolating one or more substances from the microorganism.

27. A method of treating infectious diseases in a subject which are at least concomitantly influenced by bacteria or protozoa, comprising the step of administering to the subject a substance which inhibits the synthesis of para-aminobenzoic acid from chorismic acid.

28. The method of claim 14, wherein at least some of the bacteria are Gram-positive bacteria.

29. The method of claim 27, wherein at least some of the bacteria are Gram-positive bacteria.

30. The method of claim 14, wherein the bacteria or protozoa are multiresistant to antibiotics.

31. The method of claim 27, wherein the bacteria or protozoa are multiresistant to antibiotics.

32. A microorganism capable of producing the polycyclic macrolactone of claim 1.

33. The microorganism of claim 32, wherein said microorganism is a strain or mutant of Verrucosispora.

34. The microorganism of claim 32, wherein said microorganism is Verrucosispora AB 18-032 or a mutant thereof.
